(19) United States
(12) Patent Application Publication
(10) Pub. No.: US 2016/0317438 A1
(43) Pub. Date: Nov. 3, 2016

(54) INJECTABLE SUSTAINED RELEASE INTRAOCULAR DEVICE

(71) Applicant: pSivida US, Inc., Watertown, MA (US)

(72) Inventor: Paul Ashton, Newton, MA (US)

(21) Appl. No.: 15/142,652

(22) Filed: Apr. 29, 2016

Related U.S. Application Data
(60) Provisional application No. 62/157,264, filed on May 5, 2015.
(60) Provisional application No. 62/154,491, filed on Apr. 29, 2015.

(51) Int. Cl. A61K 9/00; A61K 47/34; A61K 47/32; A61K 31/506; A61K 31/44

(52) U.S. Cl. CPC A61K 9/0051; A61K 31/506; A61K 31/44; A61K 47/32; A61K 47/34

(57) ABSTRACT

Disclosed are compositions and methods related to the use of kinase inhibitors in treating macular degeneration and/or retinal vein occlusion.

### PRIORITY CLAIM

This application claims priority to U.S. Provisional Patent Application No. 62/154,491, filed Apr. 29, 2015, and U.S. Provisional Patent Application No. 62/157,264, filed May 5, 2015, each of which is hereby incorporated by reference in its entirety.

### BACKGROUND

Age related macular degeneration (“AMD”) is the leading cause of blindness worldwide, and the World Health Organization estimates that about 14 million people are blind or severely impaired because of AMD. AMD causes the progressive loss of central vision attributable to degenerative and neovascular changes in the macula, a specialized area in the center of the retina. In general, macular degeneration can produce a slow or sudden loss of vision.

Two forms of AMD exist: dry AMD and wet AMD. Typically, AMD begins as dry AMD, which is characterized by the formation of drusen, yellow plaque-like deposits in the macula between the retinal pigment epithelium and the underlying choroid. About 15% of dry AMD patients develop wet AMD, which is characterized by the formation of new blood vessels in the choroid (choroidal neovascularization) and vision loss.

Dry macular degeneration is more common than wet AMD, with about 90% of AMD patients being diagnosed with dry AMD. The dry form of AMD may result from the aging and thinning of macular tissues, depositing of pigment in the macula, or a combination of the two processes. The wet form of the disease usually leads to more serious vision loss. With wet AMD, new blood vessels grow beneath the retina and leak blood and fluid. This leakage causes retinal cells to die and creates blind spots in central vision.

While there is no cure for AMD, treatments for wet AMD exist, such as use of anti-neovascular agents and photodynamic therapy (i.e., laser irradiation of the macula). Anti-neovascular agents for the treatment of wet AMD include agents that block the action of vascular endothelial growth factor (VEGF), thereby slowing angiogenesis. No effective treatment exists for dry AMD.

### SUMMARY

In some aspects, the invention relates to compositions and methods for treating macular degeneration by local (e.g., intraocular) administration of one or more kinase inhibitors. In some aspects, the invention relates to compositions and methods for treating retinal vein occlusion by local (e.g., intraocular) administration of one or more kinase inhibitors. In some aspects, the invention relates to sustained-release drug delivery devices that can deliver effective intraocular concentrations of the kinase inhibitor, while delivering low systemic concentrations of the kinase inhibitor, e.g., to reduce the risk of toxicity or other undesirable side effects.

### DESCRIPTION OF THE FIGURES

FIG. 1 is a graph that depicts the release of a receptor tyrosine kinase inhibitor from a device over the course of 20 days. The release rate was calculated to be 0.9161 μg per day (R²=0.9976).

### DETAILED DESCRIPTION

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The terms “bioerode”, “bioerosion”, “biodegrade”, and “biodegradation” as used herein, refer to the gradual disintegration or breakdown of a structure or enclosure over a period of time in a biological system, e.g., by one or more physical or chemical degradative processes, for example, enzymatic action, hydrolysis, ion exchange, or dissolution by solubilization, emulsion formation, or micelle formation.

The acronym “PDGF” refers to platelet-derived growth factor.

The term “preventing” is art-recognized, and when used in relation to a condition, is well understood in the art, and includes administration of a device which reduces the frequency of, or delays the onset of, symptoms of a medical condition in a subject relative to a subject which does not receive the device. Thus, prevention of macular degeneration includes, for example, reducing the number of diagnoses of macular degeneration in a treated population versus an untreated control population, and/or delaying the onset of symptoms of the macular degeneration in a treated population versus an untreated control population. Prevention of dry macular degeneration includes, for example, reducing the number of detectable drusen in a population of subjects receiving a prophylactic treatment relative to an untreated control population, and/or delaying the appearance of detectable drusen in a treated population versus an untreated control population, e.g., by a statistically and/or clinically significant amount. Prevention of vision loss includes, for example, reducing the magnitude of, or alternatively delaying, vision loss experienced by subjects in a treated population versus an untreated control population.

The term “prophylactic or therapeutic” treatment is art-recognized and includes administration to the host of one or more of the subject devices. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic (i.e., it is intended to diminish, ameliorate, or stabilize the existing unwanted condition or side effects thereof).

A “therapeutically effective amount” of a compound with respect to the subject method of treatment refers to an amount of the compound(s) in a preparation which, when administered as part of a desired dosage regimen (to a subject, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition, or to reduce the severity of disease progression.

The acronym “VEGF” refers to vascular endothelial growth factor.

### A. Kinase Inhibitors

Small molecule kinase inhibitors include afatinib, alectinib, apatinib, ASP-3026, axitinib, bafetinib, baricitinib, binimetinib, bosutinib, brigatinib, cabozantinib, canertinib, cediranib, CEP-37440, ceritinib, cobimetinib, copanlisib, crenolanib, crizotinib, CYT387, dabrafenib, damnacanthal, dasatinib, doramapimod, enterctinib, erlotinib, everolimus, filgotinib, foretinib, fostamatinib, gefitinib, grandinin, ibrutinib, icotinib, idelalisib, imatinib, IPI-145, JSI-124, lapatinib, lenvatinib, lestaurtinib, linifanib, masitinib, motesanib, mubritinib, neratinib, nilotinib, nintedanib, pacritinib, palbociclib, pazopanib, pegaptanib, perifosine, PF-06463922, ponatinib, PX-866, quizartinib, radotinib, regorafenib, ruxolitinib, selumetinib, sirolimus, sorafenib, staurosporine, sunitinib, SU6656, temsirolimus, TG101348, tivozanib, toceranib, tofacitinib, trametinib, TSR-011, vandetanib, vemurafenib, and X-396. Large molecule kinase inhibitors include aflibercept, bevacizumab, catumaxomab, panitumumab, ranibizumab, and trastuzumab.

In preferred embodiments, the kinase inhibitor is a tyrosine kinase inhibitor, such as afatinib, alectinib, apatinib, axitinib, bafetinib, baricitinib, binimetinib, bosutinib, brigatinib, cabozantinib, canertinib, cediranib, CEP-37440, ceritinib, cobimetinib, crenolanib, crizotinib, CYT387, damnacanthal, dasatinib, doramapimod, entrectinib, erlotinib, filgotinib, foretinib, fostamatinib, grandinin, gefitinib, ibrutinib, icotinib, imatinib, JSI-124, lapatinib, lestaurtinib, lenvatinib, linifanib, masitinib, motesanib, mubritinib, neratinib, nilotinib, nintedanib, pacritinib, pazopanib, pegaptanib, PF-06463922, ponatinib, quizartinib, radotinib, regorafenib, ruxolitinib, selumetinib, semaxanib, sorafenib, staurosporine, sunitinib, SU6656, TG101348, tivozanib, toceranib, tofacitinib, trametinib, TSR-011, vandetanib, vatalanib, vemurafenib, or X-396. In certain preferred embodiments, the kinase inhibitor is a receptor tyrosine kinase inhibitor.

In some embodiments, the kinase inhibitor is a multi-targeted kinase inhibitor, such as a multi-targeted receptor tyrosine kinase inhibitor.

In certain preferred embodiments, the kinase inhibitor is a VEGF receptor kinase inhibitor, PDGF receptor kinase inhibitor, and/or inflammasome inhibitor. In certain preferred embodiments, the kinase inhibitor is apatinib, axitinib, cabozantinib, cediranib, crenolanib, foretinib, lenvatinib, linifanib, masitinib, motesanib, nintedanib, pazopanib, pegaptanib, regorafenib, semaxanib, sorafenib, sunitinib, tivozanib, toceranib, vandetanib, or vatalanib. In some preferred embodiments, the kinase inhibitor is sorafenib. In some preferred embodiments, the kinase inhibitor is pazopanib. In some embodiments, the kinase inhibitor is not sunitinib.

In certain preferred embodiments, the kinase inhibitor is a BCR/Abl, Src, c-Kit, and/or ephrin receptor inhibitor. In certain preferred embodiments, the kinase inhibitor is bafetinib, bosutinib, dasatinib, imatinib, nilotinib, ponatinib, radotinib, or SU6656. In some preferred embodiments, the kinase inhibitor is dasatinib.

In certain preferred embodiments, the kinase inhibitor inhibits VEGF receptor kinase activity, e.g., by either binding to a VEGF protein or by binding to a VEGF receptor, thereby inhibiting VEGF receptor kinase activity. In some embodiments, the kinase inhibitor binds to VEGF-A, VEGF-B, VEGF-C, VEGF-D, and/or VEGF-E, e.g., thereby inhibiting VEGF receptor kinase activity. In certain embodiments, the kinase inhibitor specifically binds to and inhibits a VEGF receptor, such as VEGFR-1, VEGFR-2, and/or VEGFR-3. In certain preferred embodiments, the kinase inhibitor inhibits angiogenesis.

In certain preferred embodiments, the kinase inhibitor inhibits PDGF receptor kinase activity, e.g., by either binding to PDGF or by binding to a PDGF receptor, thereby inhibiting PDGF receptor kinase activity. In some embodiments, the kinase inhibitor binds to PDGF-A, PDGF-B, PDGF-B, PDGF-C, and/or a homodimer or heterodimer thereof, e.g., thereby inhibiting PDGF receptor kinase activity. In certain embodiments, the kinase inhibitor specifically binds to and inhibits a PDGF receptor, such as PDGFR-α and/or PDGFR-β. In certain preferred embodiments, the kinase inhibitor inhibits angiogenesis.

In certain preferred embodiments, the kinase inhibitor inhibits inflammasome activation.

In certain embodiments, the kinase inhibitor inhibits BCR/Abl, Src, c-Kit, and/or an ephrin receptor.

In certain preferred embodiments, the kinase inhibitor is a small molecule. For example, in certain preferred embodiments, the kinase inhibitor has a molecular weight of less than 1000 AMU, such as less than 600 AMU. The kinase inhibitor may have a molecular weight of less than 500 AMU. In some embodiments, the kinase inhibitor has a molecular weight between 300 AMU and 1000 AMU, such as between 300 AMU and 700 AMU, or between 300 AMU and 600 AMU.

### B. Sustained-Release Drug Delivery Devices Comprising Shells

In some aspects, the invention relates to sustained-release drug delivery devices comprising a shell, wherein the kinase inhibitor is disposed in the shell. The device may be configured such that the kinase inhibitor can diffuse through at least one permeable portion of the shell. Suitable shells are described, for example, in U.S. Pat. No. 8,871,241, hereby incorporated by reference.

The shell encloses the kinase inhibitor, keeping the kinase inhibitor localized within or in proximity to the eye. At the same time, the shell allows the kinase inhibitor to exit the shell and reach target tissues, e.g., the retina. Thus, at least a portion of the shell is permeable to the kinase inhibitor.

The device may comprise particles of the kinase inhibitor.

The shell can also provide structure to the device. The shell may be dimensionally stable and retain its shape in the absence of a core. Preferably, the shell comprises a rigid tube that retains its shape even when not filled with the kinase inhibitor. The tube may also retain its shape in the absence of the first end and second end of the shell. The tube may be bioerodible or non-bioerodible.

In some embodiments, the device comprises a single shell.

In certain embodiments, the shell is made from a tube with a member closing each end, so that the members hold the kinase inhibitor inside. The tube may have a first end and a second end. A first member may contact the first end of the tube. A second member may contact the second end of the tube. Thus, the tube, first member, and second member may enclose the kinase inhibitor within the shell. In some preferred embodiments, one end of the tube is closed with a permeable member (e.g., PVA) while the other end of the tube is closed with an impermeable member (e.g., silicone). In other preferred embodiments, both ends of the tube are closed with a permeable member.

At least one of the first member and second member may be bioerodible. At least one of the first member and second member may be non-bioerodible.

The first and second members may have about the same thickness, or they may have different thicknesses. In addition, the first and second members may have approximately the same diameter as the tube, or members may have a slightly larger or slightly smaller diameter relative to the tube, as long as they are appropriately sized to retain the kinase inhibitor in the tube. The first and second members may be formed in situ at the ends of the tube, for example, by inducing cross-linking of a polymeric layer at the ends of the tube.

In some embodiments, the device does not comprise a first member and/or second member.

Although the device of may be substantially cylindrical (i.e., it may have a tube with a circular cross-section), other geometries are possible. The device can have a cross-section that is a circle, an oval, a square, a rectangle, a hexagon, or any other shape that is suitable for containing particles inside.

In some embodiments, the length of the device (e.g., the distance between the first member and the second member) is from about 1 mm to 2 mm, 2 mm to 4 mm, 4 mm to 6 mm, 6 mm to 8 mm, 8 mm to 10 mm, 1 mm to 12 mm, 2 mm to 12 mm, or 4 mm to 12 mm. In certain embodiments, the width of the device (e.g., the diameter of the tube, or the diameter of the first and second members), is from about 0.1 mm to 0.2 mm, 0.2 mm to 0.4 mm, 0.4 mm to 0.6 mm, 0.6 mm to 0.8 mm, 0.8 mm to 1.0 mm, 1 mm to 2 mm, 2 mm to 4 mm, 4 mm to 6 mm, 6 mm to 8 mm, 8 mm to 10 mm, 1 mm to 12 mm, 2 mm to 12 mm, or 4 mm to 12 mm. The device may be shaped and sized for injection. For example, the device may be less than about 4 mm long and less than about 0.5 mm in diameter, e.g., to fit through at least one of a needle having a size from about 30 gauge to about 15 gauge or a cannula having a size from about 30 gauge to about 15 gauge. In some embodiments, the device is shaped and sized for injection through a needle or cannula having a size from about 30 gauge to about 15 gauge, such as from about 30 gauge to about 20 gauge, from about 30 gauge to about 21 gauge, from about 30 gauge to about 22 gauge, from about 30 gauge to about 23 gauge, from about 30 gauge to about 24 gauge, or from about 30 gauge to about 25 gauge. In preferred embodiments, the device is shaped and sized for injection through a needle or cannula having a size from about 30 gauge to about 22 gauge, such as from about 30 gauge to about 23 gauge, or from about 30 gauge to about 24 gauge.

In some embodiments, for example, when the device is prepared for implantation within the vitreous of the eye, the device does not exceed about 7 mm in any direction, so that the device can be inserted through a less than 7 mm incision. Thus, in some embodiments, the device does not exceed 7 mm in height or 3 mm in diameter. In preferred embodiments, the device has a length of between 1 mm and 4 mm. For example, the device may have a length of approximately 3.5 mm. In preferred embodiments, the device has a diameter of between 0.2 mm and 0.5 mm. For example, the device may have a diameter of approximately 0.37 mm.

The wall of the tube is preferably sufficiently thick to allow the tube to retain its shape in the absence of any other material. In some embodiments, the thickness of the tube walls ranges between about 0.01 mm and about 1.0 mm. In some embodiments, the tube wall is from about 0.01 mm to 0.02 mm, 0.02 mm to 0.04 mm, 0.04 mm to 0.06 mm, 0.06 mm to 0.08 mm, 0.08 mm to 1.0 mm, 1 mm to 2 mm, 2 mm to 4 mm, or 4 mm to 10 mm. Exemplary diameters for the tube of the device include 0.011″+/−0.001″ for the inner diameter and 0.0145″+/−0.001″ for the outer diameter. Exemplary diameters for the tube of the device also include 0.0061+/−0.001″ for the inner diameter and 0.0145″+/−0.001″ for the outer diameter. Exemplary diameters for the tube of the device also include 0.016″+/−0.001″ for the inner diameter and 0.018″+/−0.001″ for the outer diameter. Exemplary diameters for the tube of the device also include 0.0115″+/−0.001″ for the inner diameter and 0.0125″+/−0.001″ for the outer diameter. In preferred embodiments, the tube has a diameter of between 0.2 mm and 0.5 mm. For example, the tube may have a diameter of approximately 0.37 mm.

At least a portion of the shell may be permeable to the kinase inhibitor. “Permeable” denotes that the shell allows an effective amount of the kinase inhibitor to exit the device. Certain parts of the shell may be impermeable to the kinase inhibitor. The term “impermeable”, as used herein, means that the layer will not allow passage of the kinase inhibitor at a rate required to obtain the desired local or systemic physiological or pharmacological effect, during the period when the device delivers an effective amount of the kinase inhibitor to the patient. In some embodiments, the impermeable region has a permeability for the kinase inhibitor of less than 10%, 5%, 2%, 1%, 0.5%, 0.2%, 0.1%, 0.05%, 0.02%, or 0.01% of the permeability of the permeable region.

In some embodiments, the tube is permeable to the kinase inhibitor. In some embodiments, the first member is permeable to the kinase inhibitor. In some embodiments, the second member is permeable to the kinase inhibitor. In preferred embodiments, the tube is impermeable and one or both of the members are permeable. In some embodiments, to promote greater release of the kinase inhibitor, the tube is made of a permeable material. For instance, the tube, first member, and second member may all be permeable.

The permeability of a portion of the shell can be affected by its thickness. An impermeable member should be thick enough not to release a significant amount of kinase inhibitor, relative to a permeable region of the device. The thickness of an impermeable member can be, for example, between about 0.01 and about 2 mm, preferably between about 0.01 and about 0.5 mm, most preferably between about 0.01 and about 0.2 mm. A permeable member should preferably be thick enough to contain the kinase inhibitor in the tube, yet not so thick as to prevent release of an effective amount of the kinase inhibitor. The thickness of the permeable member may be, for example, between about 0.01 and about 2 mm, preferably between about 0.01 and about 0.5 mm, most preferably between about 0.01 and about 0.2 mm.

In certain embodiments, at least a portion of the shell is porous and the kinase inhibitor can exit the shell through the pores.

Preferably, the shell is essentially insoluble in body fluids with which the material will come in contact.

In some embodiments, the shell is substantially non-biodegradable. In other embodiments, the shell is biodegradable. In some embodiments, the shell does not substantially biodegrade in a biological environment prior to release of at least 60%, 70%, 80%, 90%, 95%, or 99% of the kinase inhibitor. In preferred embodiments, the shell does not substantially biodegrade in a biological environment prior to release of at least 90%, 95%, or 99% of the kinase inhibitor. In some embodiments, the shell substantially biodegrades in a biological environment after the release of at least 60%, 70%, 80%, 90%, 95%, or 99% of the kinase inhibitor. In preferred embodiments, the shell substantially biodegrades in a biological environment after the release of at least 90%, 95%, or 99% of the kinase inhibitor.

In preferred embodiments, the shell comprises a polymer. In particular, the tube, first member, and/or second member may be polymeric. Generally speaking, suitable biocompatible polymers for use in the subject devices include, but are not limited to, poly(vinyl acetate) (PVAC), poly(caprolactone) (PCL), ethylene vinyl acetate polymer (EVA), poly(ethylene glycol) (PEG), polyvinyl alcohol (PVA), poly(lactic acid) (PLA), poly(glycolic acid) (PGA), poly(lactic-co-glycolic acid) (PLGA), polyalkyl cyanoacrylate, polyurethane, nylons, or copolymers thereof. In polymers including lactic acid monomers, the lactic acid may be D-, L- (e.g., poly-L-lactic acid (PLLA)), or any mixture of D- and L-isomers. In some embodiments, the polymer is polyimide. In some embodiments, the polymer is PLGA that comprises lactic acid (L) and glycolic acid (G) monomers in a ratio of about 95% L and 5% G. The percentage of L may range between 80-97%. The percentage of G may range between 3-20%. In some embodiments, the polymer is heat curable, radiation curable, light (including ultraviolet) curable, evaporation curable, and/or curable by catalysis. In certain embodiments, the polymer is silicone, such as a silicone rubber, polydimethylsiloxane, or silicone-carbonate copolymer.

Certain polymers, like PVA, can be made more or less permeable by altering the degree of polymer cross-linking. Some polymers may be permeable or impermeable depending on the relative characteristics of the polymer and the drug in the drug core. For instance, a given polymer may be permeable to a small molecule but impermeable to an antibody.

Exemplary polymers suitable for construction of permeable portions of the shell include PVA and PEG.

Exemplary polymers suitable for construction of impermeable portions of the shell include nylons, polyimide, polyurethane, EVA, polyalkyl cyanoacrylate, poly(tetrafluoroethylene) (PTFE), polycarbonate (PC), poly(methyl methacrylate) (PMMA), high grades of ethylene vinyl acetate (EVA) (e.g., 9% vinyl, content), poly(lactic-co-glycolic acid) (PLGA), and polyvinyl alcohol (PVA), especially cross-linked PVA. In certain preferred embodiments, impermeable portions of the shell (e.g., the tube) comprise polyimide. In other preferred embodiments, impermeable portions of the shell (e.g., the tube) comprise poly(lactic-co-glycolic acid) or poly(lactic acid).

In certain embodiments, the shell comprises metal, such as gold, platinum, or (surgical) stainless steel. For instance, the tube may be made of metal. In some embodiments, the metal portion of the shell is impermeable to the kinase inhibitor. The metal is preferably biocompatible. In certain embodiments, the metal is biodegradable. The biocompatible and/or biodegradable metal alloy may comprise one or more of Fe (iron), Mg (magnesium), Mn (manganese), Pd (palladium), Co (cobalt), Al (aluminum), W (tungsten), B (boron), C (carbon), S (sulfur), Si (silicon), Li (lithium), Zr (zirconium), Ca (calcium), Y (yttrium), Zn (zinc). Exemplary biodegradable metals are described in H. Hermawan “Biodegradable Metals” SpringerBriefs in Materials 2012 p. 13-22 and Moravej and Martovani, “Biodegradable Metals for Cardiovascular Stent Application: Interests and New Opportunities” Int J Mol Sci. 2011; 12(7):4250-4270.

The device may include a polymeric matrix disposed in the shell. The polymeric matrix may be admixed with the kinase inhibitor (which may be present in a solid form, such as a powder, particles, or granules). The polymeric matrix may have little or no effect on the release rate of the kinase inhibitor. Alternatively, the polymeric matrix may affect the release rate of the kinase inhibitor. The matrix may be bioerodible. The matrix may buffer the pH within the shell, for example, to alter the dissolution rate of a solid drug or to protect the drug from degradation. The matrix may protect the kinase inhibitor from degradation such as degradation caused by manufacturing, storing, or using the device. The matrix may also protect the kinase inhibitor from chemical degradation and metabolism in biological fluids by controlling and limiting the interaction of the drug and fluid. For example, the matrix may inhibit or prevent the passage of chemicals, enzymes, proteins, and other materials, which could degrade the drug before it is released from the device. The matrix may comprise at least one of poly(vinyl acetate) (PVAC), poly(caprolactone) (PCL), polyethylene glycol (PEG), poly(dl-lactide-co-glycolide) (PLGA), ethylene vinyl acetate polymer (EVA), polyvinyl alcohol (PVA), poly(lactic acid) (PLA), poly(glycolic acid) (PGA), polyalkyl cyanoacrylate, polyurethane, or nylon, or a copolymer thereof. In certain preferred embodiments, the matrix comprises polyvinyl alcohol (PVA).

The device may comprise particles of the polymeric matrix, e.g., particles of the polymeric matrix admixed with the kinase inhibitor.

The materials for the tube, first member, and second member can be chosen to achieve the desired rate of release for the kinase inhibitor. For instance, a “low-flow” embodiment of the device may comprise a tube made of an impermeable material (e.g., polyimide) and a second member made of an impermeable material (e.g., silicone). The first member may be made of a permeable material (e.g., polyvinyl alcohol). The low-flow device may allow no substantial amount of the kinase inhibitor to exit the device through the tube or second member. As an example, in preferred low-flow embodiments, the tube comprises impermeable poly(dl-lactide-co-glycolide) PLGA or polyimide, the second member is made of an impermeable substance such as silicone, and the first member comprises permeable polyvinyl alcohol (PVA).

A “high-flow” embodiment of the device may comprise a tube made of an impermeable material (e.g., polyimide). The first member and second member may both be made of permeable material (e.g., polyvinyl alcohol). The kinase inhibitor may diffuse through the permeable first and second members and to exit the high-flow device. The high-flow device may prevent a substantial amount of the kinase inhibitor from exiting the device through the tube. As an example, in preferred embodiments of a high-flow device, the tube comprises impermeable poly(dl-lactide-co-glycolide) PLGA or polyimide, and the first and second members comprise permeable polyvinyl alcohol (PVA).

The kinase inhibitor may diffuse in the direction of lower chemical potential, i.e., toward the exterior surface of the device. Release of the kinase inhibitor from the device may be controlled by several factors. Release may be influenced, for example, by the kinase inhibitor's dissolution rate and passage through the shell. The device shape, size, and materials can be chosen to achieve a desired release rate of the kinase inhibitor. In some embodiments, the release rate is determined primarily by dissolution of the kinase inhibitor. In other embodiments, the release rate is determined primarily by the permeability of the shell. In some embodiments, the release rate is significantly affected by both dissolution of the kinase inhibitor and the permeability of the shell.

The rate of diffusion of the kinase inhibitor through the shell may be determined, for instance, via diffusion cell studies carried out under sink conditions. In diffusion cell studies carried out under sink conditions, the concentration of drug in the receptor compartment is essentially zero when compared to the high concentration in the donor compartment.

It will be appreciated that a material may be permeable to a drug and also substantially control the rate at which the drug diffuses or otherwise passes through the material. Consequently, a permeable portion of the shell may also be release-rate-limiting or release-rate-controlling, and the permeability of such a membrane may be one of the most significant factors controlling the release rate for a device.

In preferred embodiments, the device releases the kinase inhibitor at a rate that is essentially constant over time (i.e., zero-order kinetics). Zero-order release is desirable when the goal is to maintain a substantially constant amount of the kinase inhibitor to the patient over a sustained period.

It will be appreciated that many techniques may be employed to preform a tube useful for making the injectable drug delivery devices described herein, such as those described in U.S. Pat. No. 8,871,241, herein incorporated by reference.

In preferred embodiments, the device is a sustained-release drug delivery device for insertion into the vitreous of an eye, comprising a shell and a kinase inhibitor disposed in the shell; wherein the device is shaped and sized for injection through a needle or cannula having a size from about 30 gauge to about 22 gauge; the device is configured to release the kinase inhibitor at a rate of less than 10 μg per day; and the device is configured to maintain a therapeutically effective concentration of the kinase inhibitor in the eye for a period of time between 2 weeks and 4 years.

### C. Administering a Device

In preferred embodiments, the devices are administered to prevent or treat a condition or disease of the eye.

In certain embodiments, the devices are administered to prevent or treat macular degeneration in a subject, e.g., age-related macular degeneration (“AMD”), such as dry AMD and wet AMD. The device may be administered to prevent the death of retinal pigment epithelial cells. The device may be administered to prevent Alu-RNA induced cytotoxicity. The device may be administered to inhibit P2X7 activation. The device may be administered to inhibit caspase-1 activation. The device may be administered to inhibit angiogenesis. In some embodiments, the devices are administered to prevent or treat vision loss in a subject, such as vision loss associated with macular degeneration. The device may be administered to prevent geographic atrophy in an eye. The device may be administered to prevent or delay the progression of dry AMD to wet AMD.

In some embodiments, the devices are administered to prevent or treat retinal vein occlusion in a subject, e.g., central retinal vein occlusion (“CRVO”) or branch retinal vein occlusion (“BRVO”). The devices may be administered to prevent or treat non-ischemic retinal vein occlusion or ischemic retinal vein occlusion.

The various embodiments provided herein are generally provided to deliver a therapeutically effective concentration of a kinase inhibitor locally, e.g., to the eye of a subject. In certain embodiments, the devices of the invention may be delivered to any site in or on the eye. For example, devices of the invention may be used on the surface of the eye or may be implanted within the eye. The device may be administered, for example, intravitreally or subretinally. In certain embodiments, a device of the invention is delivered to the surface of the eye or within the eye such as within the uveal tract of the eye. In preferred embodiments, a device of the invention is delivered within the vitreous of the eye.

In certain embodiments, the method for treating an ocular condition comprises disposing the device on the surface of the eye or within the eye such as within the vitreous or aqueous of the eye. In certain embodiments, the device is injected or surgically inserted within the eye of the subject. In preferred embodiments, the device is injected within the eye of the subject, e.g., into the vitreous of the eye.

In some aspects, the invention relates to the local administration of a device. In preferred embodiments, administering a device comprises injecting the device. Administering a device may comprise inserting the device into the eye, such as inserting the device into the aqueous humor or, preferably, into the vitreous humor of the eye. Administering a device may comprise surgically implanting the device into or onto the eye, such as a scleral implant, subconjunctival implant, suprachoroidal implant, suprascleral implant, or intravitreal implant. The device can be surgically implanted into the eye of the subject, for example the vitreous of the eye, under the retina, and onto the sclera. When the kinase inhibitor acts on the eye, the device can gradually release the kinase inhibitor to the eye, avoiding painful repeated administrations of a different formulation of the kinase inhibitor.

In some embodiments the device is configured to maintain a therapeutically effective concentration of the kinase inhibitor at the site for at least 1, 2, 3, 4, 5, 6, 7, or 8 weeks. The device may be configured to maintain a therapeutically effective concentration of the kinase inhibitor at the site for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 months. For example, the device may be configured to maintain a therapeutically effective concentration of the kinase inhibitor at the site for at least 1, 2, 3, or 4 years. In certain preferred embodiments, the device is configured to maintain a therapeutically effective concentration of the kinase inhibitor at the site for 2, 3, 4, 5, 6, 7, 8, 9, 10, 12, 18, 20, 24, 30, or 36 months, or 1, 2, 3, or 4 years. In certain preferred embodiments, the device is configured to maintain a therapeutically effective concentration of the kinase inhibitor at the site for a period of time between 2 weeks and 4 years, such as between 2 months and 3 years, or between 6 months and 30 months. In some preferred embodiments, the device is configured to maintain a therapeutically effective concentration of the kinase inhibitor at the site for a period of time between 1 month and 36 months, such as between 2 months and 12 months, or between 4 months and 12 months. In some embodiments, the device is configured to maintain a therapeutically effective concentration of the kinase inhibitor at the site for a period of about 6 months.

In preferred embodiments, the device releases the kinase inhibitor at a rate of less than 10 μg per day, such as less than 9 μg per day, less than 8 μg per day, less than 7 μg per day, less than 6 μg per day, less than 5 μg per day, less than 4 μg per day, less than 3 μg per day, less than 2 μg per day, or less than 1 μg per day. The device may release the kinase inhibitor at a rate of about 0.5 μg per day, about 0.6 μg per day, about 0.7 μg per day, about 0.8 μg per day, about 0.9 μg per day, about 1 μg per day, about 1.2 μg per day, about 1.5 μg per day, about 2 μg per day, about 3 μg per day, about 4 μg per day, about 5 μg per day, about 6 μg per day, about 7 μg per day, or about 8 μg per day. The device may release the kinase inhibitor at a rate of between about 0.1 μg per day and about 10 μg per day, such as between about 0.2 μg per day and about 8 μg per day, between about 0.3 μg per day and about 6 μg per day, or between about 0.4 μg per day and about 4 μg per day.

In some embodiments, more than one device is administered to the subject.

In preferred embodiments, the method is a method for treating an eye disease in a human subject, comprising administering to the subject a sustained-release drug delivery device comprising a shell and a kinase inhibitor disposed in the shell; wherein the device is shaped and sized for injection through a needle or cannula having a size from about 30 gauge to about 22 gauge; the device is configured to release the kinase inhibitor at a rate of less than 10 μg per day; and the device is configured to maintain a therapeutically effective concentration of the kinase inhibitor in the eye for a period of time between 2 weeks and 4 years. Administering preferably comprises injecting the device into the eye, preferably the vitreous of the eye.

### D. Subjects

The subject may be selected from rodents, lagomorphs, ovines, porcines, canines, felines, equines, bovines, and primates. In preferred embodiments, the subject is a human or rabbit. The subject may have an eye disease.

In some embodiments, the subject has macular degeneration, such as age-related macular degeneration (“AMD”) or dry macular degeneration, or retinal vein occlusion (“RVO”). In some embodiments, the subject has dry AMD. The subject may have geographic atrophy, such as geographic atrophy that impairs the vision of the subject. The subject may be at risk for developing geographic atrophy. The device may be administered to prevent geographic atrophy. The subject may have vision loss or the subject may be at risk for developing vision loss, e.g., due to macular degeneration. The subject may be at risk for developing wet AMD. The device may be administered to prevent the development of wet AMD.

In some embodiments, the subject has retinal vein occlusion (“RVO”), e.g., central retinal vein occlusion (“CRVO”) or branch retinal vein occlusion (“BRVO”). The subject may have ischemic retinal vein occlusion or non-ischemic retinal vein occlusion. The subject may have vision loss or the subject may be at risk for developing vision loss, e.g., due to retinal vein occlusion. The subject may have glaucoma or the subject may be at risk for developing glaucoma, e.g., due to retinal vein occlusion. The subject may have macular edema or the subject may be at risk for developing macular edema, e.g., due to retinal vein occlusion.

### EXEMPLIFICATION

### Example 1

### Method of Manufacturing a Device Comprising a Shell (Sorafenib)

400 mg of sorafenib is granulated with 200 μl of freshly prepared 10% PVA solution and the granulation is air-dried. PVA solution is prepared from 98 mole % hydrolyzed polyvinyl alcohol, MW˜78,000 (Polysciences, Inc.). The dried granulation is ground to fine particles. 360 mg of granulation is blended with 1.0 mg of magnesium stearate. 45 pellets (2.0 mm Ø, 6.30±0.2 mg) are prepared using a hand tablet press. The pellets are dipping coated in 5.0% PVA solution, air dried, and then heat treated at 135° C. for 3 hours.

One folded suture (Ethicon, Perma-Hand silk) is placed into each 3 mm-long polyimide tube and then a sorafenib pellet is also placed into the tube. Both ends of the tube are covered with silicone adhesive and a polyimide suture tag is attached to the implant with silicone adhesive. The implants are cured at room temperature for 24 hours.

The suture is pulled from the implant to leave diffusion windows at the top and bottom of the implants. The bottom windows are sealed with silicone adhesive leaving windows at one end of the implant. The implants are again cured at room temperature for 72 hours.

Implants are visually inspected under a microscope for integrity. A total of 30 implants are prepared. Four implants are used to test the in vitro release (in 5 mL PBS), and three implants are used to determine drug content (see Example 2, infra).

Each implant is placed in sterile micro-centrifuge tube and then packaged in a Chex-all pouch (Propper). The packaged units are gamma irradiated at a dose of 28 kGy.

### Example 2

### Performance of a Device Comprising a Shell (Sorafenib)

Each sorafenib implant assayed for content is cut in half and placed in a 25 mL volumetric flask. 20 mL deionized water is added to the flask which is sonicated (3×20 minutes). Sorafenib drug content is measured by HPLC.

Implants assayed for release rate are placed individually in 10 mL glass tubes, and 5 mL PBS is added to each tube. The tubes are incubated in a water bath at 37° C. Samples are taken at 12 to 24 hour intervals, and the release medium is replaced with fresh PBS. The amount of sorafenib released is measured quantitatively by HPLC. The in vitro release rate is tested for 5 days, and the average release rate is determined from the cumulative release versus time.

### Example 3

### Method of Manufacturing a Device Comprising a Shell (Dasatinib)

400 mg of dasatinib is granulated with 200 μl of freshly prepared 10% PVA solution and the granulation is air-dried. PVA solution is prepared from 98 mole % hydrolyzed polyvinyl alcohol, MW˜78,000 (Polysciences, Inc.). The dried granulation is ground to fine particles. 360 mg of granulation is blended with 1.0 mg of magnesium stearate. 45 pellets (2.0 mm Ø, 6.30±0.2 mg) are prepared using a hand tablet press. The pellets are dipping coated in 5.0% PVA solution, air dried, and then heat treated at 135° C. for 3 hours.

One folded suture (Ethicon, Perma-Hand silk) is placed into each 3 mm-long polyimide tube and then a dasatinib pellet is also placed into the tube. Both ends of the tube are covered with silicone adhesive and a polyimide suture tag is attached to the implant with silicone adhesive. The implants are cured at room temperature for 24 hours.

The suture is pulled from the implant to leave diffusion windows at the top and bottom of the implants. The bottom windows are sealed with silicone adhesive leaving windows at one end of the implant. The implants are again cured at room temperature for 72 hours.

Implants are visually inspected under a microscope for integrity. A total of 30 implants are prepared. Four implants are used to test the in vitro release (in 5 mL PBS), and three implants are used to determine drug content (see Example 4, infra).

Each implant is placed in sterile micro-centrifuge tube and then packaged in a Chex-all pouch (Propper). The packaged units are gamma irradiated at a dose of 28 kGy.

### Example 4

### Performance of a Device Comprising a Shell (Dasatinib)

Each dasatinib implant assayed for content is cut in half and placed in a 25 mL volumetric flask. 20 mL deionized water is added to the flask which is sonicated (3×20 minutes). Dasatinib drug content is measured by HPLC.

Implants assayed for release rate are placed individually in 10 mL glass tubes, and 5 mL PBS is added to each tube. The tubes are incubated in a water bath at 37° C. Samples are taken at 12 to 24 hour intervals, and the release medium is replaced with fresh PBS. The amount of dasatinib released is measured quantitatively by HPLC. The in vitro release rate is tested for 5 days, and the average release rate is determined from the cumulative release versus time.

### Example 5

### Method of Manufacturing a Device Comprising a Shell (Pazopanib)

400 mg of pazopanib is granulated with 200 μl of freshly prepared 10% PVA solution and the granulation is air-dried. PVA solution is prepared from 98 mole % hydrolyzed polyvinyl alcohol, MW˜78,000 (Polysciences, Inc.). The dried granulation is ground to fine particles. 360 mg of granulation is blended with 1.0 mg of magnesium stearate. 45 pellets (2.0 mm Ø, 6.30±0.2 mg) are prepared using a hand tablet press. The pellets are dipping coated in 5.0% PVA solution, air dried, and then heat treated at 135° C. for 3 hours.

One folded suture (Ethicon, Perma-Hand silk) is placed into each 3 mm-long polyimide tube and then a pazopanib pellet is also placed into the tube. Both ends of the tube are covered with silicone adhesive and a polyimide suture tag is attached to the implant with silicone adhesive. The implants are cured at room temperature for 24 hours.

The suture is pulled from the implant to leave diffusion windows at the top and bottom of the implants. The bottom windows are sealed with silicone adhesive leaving windows at one end of the implant. The implants are again cured at room temperature for 72 hours.

Implants are visually inspected under a microscope for integrity. A total of 30 implants are prepared. Four implants are used to test the in vitro release (in 5 mL PBS), and three implants are used to determine drug content (see Example 6, infra).

Each implant is placed in sterile micro-centrifuge tube and then packaged in a Chex-all pouch (Propper). The packaged units are gamma irradiated at a dose of 28 kGy.

### Example 6

### Performance of a Device Comprising a Shell (Pazopanib)

Each pazopanib implant assayed for content is cut in half and placed in a 25 mL volumetric flask. 20 mL deionized water is added to the flask which is sonicated (3×20 minutes). Pazopanib drug content is measured by HPLC.

Implants assayed for release rate are placed individually in 10 mL glass tubes, and 5 mL PBS is added to each tube. The tubes are incubated in a water bath at 37° C. Samples are taken at 12 to 24 hour intervals, and the release medium is replaced with fresh PBS. The amount of pazopanib released is measured quantitatively by HPLC. The in vitro release rate is tested for 5 days, and the average release rate is determined from the cumulative release versus time.

### Example 7

### Release Rate for a Device Comprising a Shell

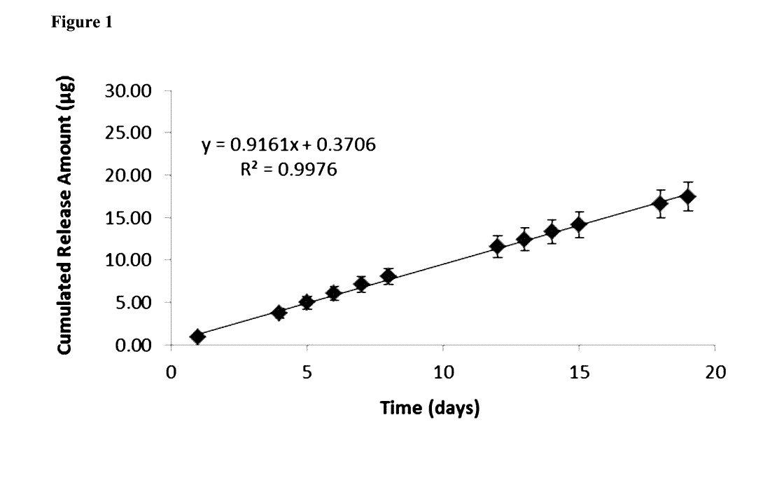

A device comprising a receptor tyrosine kinase inhibitor was constructed using methods similar to those described in Examples 1, 3, and 5. The release of the kinase inhibitor was monitored using methods similar to those described in Examples 2, 4, and 6. The device released the kinase inhibitor at a constant rate of about 0.92 μg per day over the course of 20 days (FIG. 1).

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

1. A method for treating an eye disease in a subject, comprising administering to the subject a sustained-release drug delivery device comprising:
a shell; and
a kinase inhibitor disposed in the shell,
wherein the device is shaped and sized for injection through a needle or cannula having a size from about 30 gauge to about 22 gauge.

2. (canceled)

3. The method of claim 1, wherein the kinase inhibitor is a VEGF receptor kinase inhibitor, PDGF receptor kinase inhibitor, or inflammasome inhibitor.

4. The method of claim 1, wherein the kinase inhibitor is apatinib, axitinib, cabozantinib, cediranib, crenolanib, foretinib, lenvatinib, linifanib, masitinib, motesanib, nintedanib, pazopanib, pegaptanib, regorafenib, semaxanib, sorafenib, sunitinib, tivozanib, toceranib, vandetanib, or vatalanib.

5. The method of claim 4, wherein the kinase inhibitor is sorafenib or pazopanib.

6. The method of claim 1, wherein the kinase inhibitor is bafetinib, bosutinib, dasatinib, imatinib, nilotinib, ponatinib, radotinib, or SU6656.

7. The method of claim 6, wherein the kinase inhibitor is dasatinib.

8. The method of claim 1, wherein the kinase inhibitor is admixed with a polymeric matrix.

9. The method of claim 8, wherein the polymeric matrix comprises one or more polymers selected from poly(vinyl acetate), poly(caprolactone), polyethylene glycol, poly(dl-lactide-co-glycolide), ethylene vinyl acetate polymer, polyvinyl alcohol, poly(lactic acid), poly(glycolic acid), polyalkyl cyanoacrylate, polyurethane, and nylon, and a copolymer thereof.

10. The method of claim 9, wherein the polymeric matrix comprises polyvinyl alcohol (PVA).

11. The method of claim 1, wherein the shell comprises a tube having first and second ends, and the kinase inhibitor is disposed in the tube.

12. The method of claim 11, wherein the tube comprises one or more polymers selected from polyimide, poly(vinyl acetate), poly(caprolactone), polyethylene glycol, poly(dl-lactide-co-glycolide), ethylene vinyl acetate polymer, polyvinyl alcohol, poly(lactic acid), poly(glycolic acid), polyalkyl cyanoacrylate, polyurethane, polyimide, and nylon, and a copolymer thereof.

13-15. (canceled)

16. The method of claim 11, wherein the tube is impermeable to the kinase inhibitor.

17. The method of claim 11, wherein:
the shell further comprises a first member positioned at the first end of the tube and a second member positioned at the second end of the tube; and
the first member is permeable to the passage of the kinase inhibitor.

18-19. (canceled)

20. The method of claim 17, wherein the first member or the second member comprises one or more polymers selected from poly(vinyl acetate), poly(caprolactone), polyethylene glycol, poly(dl-lactide-co-glycolide), ethylene vinyl acetate polymer, polyvinyl alcohol, poly(lactic acid), poly(glycolic acid), polyalkyl cyanoacrylate, polyurethane, and nylon, and a copolymer thereof.

21. The method of claim 20, wherein the first member or the second member comprises polyvinyl alcohol (PVA).

22. The method of claim 17, wherein the second member is permeable to the passage of the kinase inhibitor.

23-24. (canceled)

25. The method of claim 17, wherein the second member is impermeable to the passage of the kinase inhibitor.

26. The method of claim 25, wherein the second member comprises silicone.

27. The method of claim 11, wherein the tube has a length of between 1 mm and 4 mm.

28. The method of claim 11, wherein the tube has a diameter of between 0.2 mm and 0.5 mm.

29. The method of claim 1, wherein the device is bioerodible.

30-31. (canceled)

32. The method of claim 1, wherein administering the device comprises inserting the device into an eye of the subject.

33. (canceled)

34. The method of claim 32, wherein administering the device comprises injecting the device into the vitreous of the eye.

35. (canceled)

36. The method of claim 1, wherein the device is configured to maintain a therapeutically effective concentration of the kinase inhibitor in the eye for at least 1 week, at least 1 month, or at least 6 months.

37-38. (canceled)

39. The method of claim 36, wherein the device is configured to maintain a therapeutically effective concentration of the kinase inhibitor in the eye for a period of time between 2 weeks and 4 years, between 2 months and 3 years, between 6 months and 30 months, between 1 month and 12 months, or between 4 months and 12 months.

40-43. (canceled)

44. The method of claim 1, wherein the device is configured to release the kinase inhibitor at a rate of less than 10 μg per day.

45. The method of claim 44, wherein the device is configured to release the kinase inhibitor at a rate of about 0.1 μg per day to about 10 μg per day or a rate of about 0.4 μg per day to about 4 μs per day.

46. (canceled)

47. The method of claim 1, wherein the subject is selected from rodents, lagomorphs, ovines, porcines, canines, felines, equines, bovines, and primates.

48. The method of claim 47, wherein the subject is a human.

49. The method of claim 1, wherein the subject has age-related macular degeneration, dry macular degeneration, or wet macular degeneration.

50-54. (canceled)

55. The method of claim 1, wherein the subject has geographic atrophy, the subject is at risk of developing geographic atrophy, the subject has vision loss; the subject is at risk of developing vision loss, or the subject has ischemic or non-ischemic retinal vein occlusion.

56-59. (canceled)

60. A sustained-release drug delivery device for insertion into the vitreous of an eye, comprising:
a shell; and
a kinase inhibitor disposed in the shell;
wherein:
the device is shaped and sized for injection through a needle or cannula having a size from about 30 gauge to about 22 gauge;
the device is configured to release the kinase inhibitor at a rate of less than 10 μg per day; and
the device is configured to maintain a therapeutically effective concentration of the kinase inhibitor in the eye for a period of time between 2 weeks and 4 years.

61. The device of claim 60, wherein the kinase inhibitor is apatinib, axitinib, cabozantinib, cediranib, crenolanib, foretinib, lenvatinib, linifanib, masitinib, motesanib, nintedanib, pazopanib, pegaptanib, regorafenib, semaxanib, sorafenib, sunitinib, tivozanib, toceranib, vandetanib, or vatalanib.

62. The device of claim 61, wherein the kinase inhibitor is sorafenib.

63. The device of claim 61, wherein the kinase inhibitor is pazopanib.

64. The device of claim 60, wherein the kinase inhibitor is bafetinib, bosutinib, dasatinib, imatinib, nilotinib, ponatinib, radotinib, or SU6656.

65. The device of claim 64, wherein the kinase inhibitor is dasatinib.

66. A method for treating an eye disease in a human subject, comprising administering to the subject a sustained-release drug delivery device comprising:
a shell; and
a kinase inhibitor disposed in the shell;
wherein:
the device is shaped and sized for injection through a needle or cannula having a size from about 30 gauge to about 22 gauge;
the device is configured to release the kinase inhibitor at a rate of less than 10 μg per day; and
the device is configured to maintain a therapeutically effective concentration of the kinase inhibitor in the eye for a period of time between 2 weeks and 4 years.

67. The method of claim 66, wherein the kinase inhibitor is apatinib, axitinib, cabozantinib, cediranib, crenolanib, foretinib, lenvatinib, linifanib, masitinib, motesanib, nintedanib, pazopanib, pegaptanib, regorafenib, semaxanib, sorafenib, sunitinib, tivozanib, toceranib, vandetanib, or vatalanib.

68. The method of claim 67, wherein the kinase inhibitor is sorafenib.

69. The method of claim 67, wherein the kinase inhibitor is pazopanib.

70. The method of claim 66, wherein the kinase inhibitor is bafetinib, bosutinib, dasatinib, imatinib, nilotinib, ponatinib, radotinib, or SU6656.

71. The method of claim 70, wherein the kinase inhibitor is dasatinib.
